(19) United States
(12) Patent Application Publication
(10) Pub. No.: US 2022/0110313 A1
(43) Pub. Date: Apr. 14, 2022

(54) Biological Control System Comprising Predator Acarians In A Case

(71) Applicant: Bioline France, Paris (FR)

(72) Inventors: Simon Foster, Dedham Essex (GB); Maria Riera Turro, Ipswich Suffolk (GB); Maxime Ferrero, Bar Sur Loup (FR); Jacques Frandon, Antibes (FR)

(73) Assignee: Bioline France, Paris (FR)

(21) Appl. No.: 17/264,501

(22) PCT Filed: Jul. 31, 2019
(86) PCT No.: PCT/EP2019/070703

§ 371 Date: Jan. 29, 2021

(30) Foreign Application Priority Data
Jul. 31, 2018 (FR) 1857147

(51) Int. Cl. A01M 99/00; A01K 67/033

(52) U.S. Cl. CPC A01M 99/00; A01K 67/033

(57) ABSTRACT

The invention relates to a system for the controlled spreading of predator acarians, wherein the system comprises at least one case, the case having at least one orifice suitable for predator acarians to pass through from the inside of the case to the outside of the case, the system also comprising, in each case, predator acarians, prey acarians of said predator acarians, a substrate for the prey acarians and for the predator acarians and preferentially a source of food for the prey acarians, the system being characterized in that the weight of the substrate is less than twice the total weight of the predator acarians and of the prey acarians.

### FIELD OF THE INVENTION

The invention belongs to the field of biological control and, in particular, systems and methods for biological control implementing biological control agents, in particular mites.

### STATE OF THE ART

The use of biological control agents is a method of controlling pests, pathogens or weeds by means of natural antagonist organisms thereof, such as phytophages (in the case of weeds), parasitoids (in the case of arthropods, etc.), predators (in the case of nematodes, arthropods, vertebrates, molluscs, bats, etc.) or pathogens (in the case of viruses, bacteria, fungi, etc.).

The use of these biological control agents allows reducing the use of pesticides.

Usually, biological control agents are introduced into crops to be protected in the form of compositions presented in bags comprising predators accompanied by food for them to feed themselves for a certain period, or in bulk, in the form of bottles that allow depositing variable quantities of predators on plants or on the soil. The predators then colonize the crops, where they feed on the target prey.

In particular, crops can be protected from one or more types of mite, designated by “prey mite”. It is known to spread predator mites onto crops, so as to protect them from prey mites.

In particular, WO 2011/104002 describes a system for the controlled spread of predator mites. The system comprises a capsule comprising a substrate, predator mites and prey mites. The population of predator mites feeding on the prey mites can grow over time, and leave the capsule by orifices, so as to colonize the crop. However, the duration of spreading predator mites, as well as the quantity of predator mites spread are limited.

To this end, WO 2013/043050 describes a system presenting an optimized spreading duration. The system comprises a container with an orifice suitable for the escape of predator mites. The duration of spread is optimized by introducing into the container a substrate, a population of predator mites and a population of prey mites, each having a growth rate below 0.28, the growth rate of the prey mite population being greater than the growth rate of the predator mite population. Thus, the system allows spreading predator mites for 6 weeks, for example. Moreover, this system allows spreading 400 mites per gram of substrate introduced into the container throughout the service life of the system. However, the duration of spreading predator mites, as well as the quantity of predator mites spread are still limited. There is a need for improved means for increasing the service life of biological control agents in situ, therefore permitting a lasting installation of these agents in greenhouses or in fields.

### SUMMARY OF THE INVENTION

One goal of the invention is to propose a solution for increasing the number of predator mites spread by a system for controlled spread of predator mites, relative to the weight of the compounds introduced into the system.

Another goal of the invention is to increase the service life of a system for controlled mite spread.

These goals are at least partially achieved in the context of the present invention via a system for controlled spread of predator mites, the system comprising at least one rigid case, each case having at least one orifice suitable for the passage of predator mites from the inside of the case to the outside of the case, the system also comprising, in each case, predator mites, prey mites for said predator mites, a substrate for prey mites and predator mites and, preferentially, a food source for prey mites, the system being characterized in that the weight of the substrate is less than twice, in particular less than 30% and preferentially less than 10% of the total weight of predator mites and prey mites in each case.

Thus, the inventors discovered that the introduction into a case of a high weight of predator mites and prey mites compared to the weight of the substrate unexpectedly favors the growth and spreading kinetics of the predator mites in the system.

The invention is advantageously supplemented by the following characteristics, taken individually or in any one of the technically-possible combinations thereof:
the ratio between the number of predator mites and prey mites in the case is comprised between 10⁻⁴ and 1, and preferentially comprised between 4×10⁻³ and 0.5;
the case is partially filled, so as to form an air phase, the volume of the air phase being greater than 20% of the total volume of the case, preferentially greater than 50% of the total volume of the case, and preferentially greater than 70% of the total volume of the case;
the case comprises at least two orifices;
the case is formed by a wall, the material of the wall being chosen from a material comprising fibers, the fibers comprising cellulose, and a polymer material, preferentially a biopolymer;
the case has an internal volume comprised between 0.1 mL and 30 mL, preferentially between 0.1 mL and 10 mL and preferentially between 0.5 mL and 2 mL;
each orifice has a diameter l comprised between 0.5 mm and 5 mm, preferentially between 1 mm and 2 mm;
the case is a capsule;
the predator mites are at least chosen from the Phytoseiidae, laelapidae, macrochelidae and cheyletidae families and the prey mites are at least chosen from the carpoglyphidae, pyroglyphidae, glyciphagidae, acaridae, suidasidae and cortoglyphidae families;
the predator mites are at least chosen from Neoseiulus cucumeris, Amblyseius swirskii, Transeius montdorensis, Amblyseius andersoni and Neoseiulus californicus, and the prey mites are at least chosen from Tyrolicus casei, Tyrophagus putrescentiae, Thyreophagus entomophagus, Acarus siro, Carpoglyphus lactis and Lepidoglyphus destructor;
preferentially, the predator mites are Neoseiulus cucumeris and the prey mites are Tyrophagus putrescentiae.

The invention also relates to a manufacturing method for the system, comprising a step of introducing a substrate, predator mites and prey mites into the case, the weight of the substrate being less than twice, notably less than 30% and preferentially less than 10% of the total weight of predator mites and prey mites.

Advantageously, the ratio between the number of predator mites and prey mites in the case is comprised between 10⁻⁴ and 1, and preferentially comprised between 4×10⁻³ and 0.5.

The invention also has for a subject the use of a system described previously for biological control.

The invention also has for a subject a method for biological control comprising a step of depositing at least one system described previously into a plant crop.

### PRESENTATION OF THE DRAWINGS

Other characteristics and advantages will appear from the following description, which is purely illustrative and non-limiting and should be read with regard to the attached figures, in which:

FIG. 1 schematically illustrates a case, in particular a capsule, comprising substrate, prey mites and predator mites;

FIG. 2A and FIG. 2B are diagrams illustrating the growth kinetics of the predator mites for different proportions of predator mites, prey mites and substrates introduced into the case;

FIG. 3A and FIG. 3B are diagrams illustrating the growth kinetics of the predator mites for different degrees of filling the case;

FIG. 4a and FIG. 4b are diagrams illustrating the spreading kinetics of the predator mites on the ground by a system according to the invention.

### DEFINITIONS

“Case” designates a box or rigid protective covering, i.e., having a wall allowing the case to be self-supporting. Due to the rigidity of the case wall, the case is designed to receive the mite populations, the substrate and, preferentially, food for the prey mites without being deformed. The rigid case 3 may be chosen from a rigid capsule, a rigid bag, a tube, a rigid container and a rigid box. “Capsule” designates a particular type of rigid case, having at least one rounded wall. A capsule can be in the overall form of a sphere. A tube can have at least one orifice at one end of the tube, and preferentially an orifice at each end of the tube.

The wall of the case 3 preferentially has a flexural stiffness greater than 0.2 mN·m, notably greater than 0.5 mN·m, and preferentially greater than 1 mN·m. The rigidity and flexural stiffness S are considered as being of the same magnitude, defined by formula 1:

$$S = \frac{{Ed}^{ 3}}{12} \qquad ( 1 )$$

where E is the modulus of elasticity of the wall material, and d is the thickness of the wall.

Preferentially, the wall forming the rigid case is closed while being pierced with orifices. “Closed” designates a surface whose orifices correspond to a surface area of less than 1% of the total outer surface area of the container.

The surface area of these orifices is less than 1% of the total surface area of the wall, which allows the surface to be considered “closed”.

Preferentially, the wall of at least a part of the case has a rounded shape.

### DETAILED DESCRIPTION OF THE INVENTION

General Architecture of the System for Controlled Mite Spread

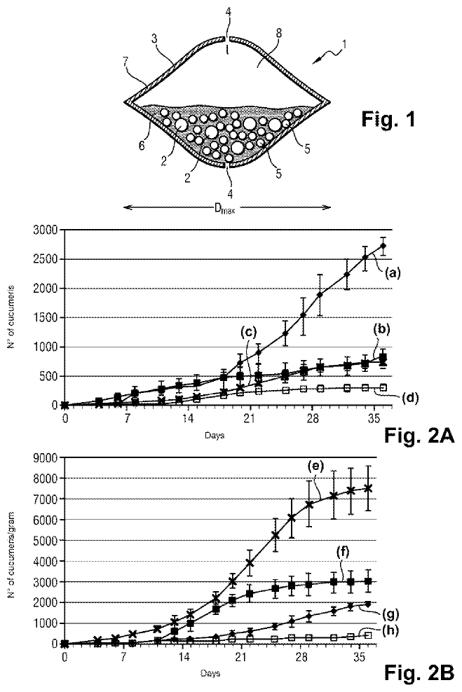

In reference to FIG. 1, system 1 comprises a case 3. The case 3 is formed by a wall 7. The material and dimensions of wall 7 are designed to make the case 3 rigid. The material of wall 7 can comprise fibers, the fibers comprising cellulose. The material of wall 7 can also be a polymer material, and preferentially a biopolymer. Thus, system 1 can be biodegradable and its use does not lead to pollution of the crops to be protected while exhibiting an appropriate permeability for the growth of predator mites. Wall 7 can also be made of expanded polymer. Advantageously, and for a given wall thickness, the water vapor permeability π of wall 7 can be less than 5 g·m⁻¹·H⁻¹·mmHg⁻¹ and preferentially less than 1 g·m⁻¹·H⁻¹·mmHg⁻¹. The case 3 has at least one orifice 4 suitable for the passage of predator mites from the inside of the case 3 to the outside of the case 3. Orifice(s) 4 are created through wall 7. Preferentially, the case 3 has at least two orifices 4, and preferentially two orifices 4. Orifices 4 can be arranged in opposition, for example, in wall 7. Thus, the gas phase inside the case 3, in particular air, can be renewed more homogenously. Orifice 4 has a diameter l comprised between 0.5 mm and 5 mm, preferentially between 1 mm and 2 mm. “Diameter” means the maximum size of orifice 4. The case 3 has an internal volume comprised between 0.1 mL and 10 mL, preferentially between 0.5 mL and 2 mL. The case 3 can have a spherical, ovoid or pancake shape. Preferentially, diameter Dₘₐₓ of the case 3 is comprised between 0.5 cm and 10 cm, preferentially between 1 cm and 3 cm. The dimensions of the case 3 (i.e., for example, the choice of internal volume, diameter, arrangement of orifices 4, diameter of orifices 4 and thickness of wall 7) as well as the choice of material for wall 7 lead to a humidity level in the case 3 and thermal transfer between the case 3 and the external environment conducive to the growth of predator mites.

The case 3 comprises at least two mite populations: a prey mite population 5 and a predator mite population 2 for prey mites 5. The case 3 comprises a substrate 6, as well as, optionally, a food source for prey mites 5. Substrate 6 is a porous solid phase that is suited to the shape of the case 3, in which the different mite populations can move and grow. Substrate 6 can be, for example, formed by different types of grains, for example bran, and/or by mineral particles. The food source for the prey mites is different from substrate 6, although for some substrate 6 and prey mite 5 pairings, substrate 6 can represent an additional food source for prey mites 5. Prey mite food source 5, distinct from substrate 6, can comprise, for example, yeast such as brewer's yeast, oils, for example oils comprising amino acids, and/or pollen.

Substrate 6 is known, in the prior art, to promote the growth of both mite populations. Indeed, substrate 6 can mimic their natural environment. The inventors have discovered, that contrary to prior belief, the growth of predator mites and the capacity of system 1 to emit predator mites 2 are enhanced by a low weight of substrate 6 compared to the cumulative weight of predator mites 2 and prey mites 5, particularly when the weight of substrate 6 is less than twice, especially less than 30% and preferentially less than 10% of the total weight of predator mites 2 and prey mites 5. Indeed, the reduction of the ratio between the weight of substrate 6 and the total weight of mites facilitates the interaction and predation between the two mite populations. Moreover, the combination of a case 3 and a weight of substrate 6 chosen so that the weight of substrate 6 is less than twice, particularly less than 30% and preferentially less than 10% of the total weight of predator mites 2 and prey mites 5, surprisingly allows moisture to be absorbed inside the case 3 during the growth of the mites. The ratio between the weight of substrate 6 and the weight of the mites allows moisture to be absorbed in the case 3. The weight of substrate 6 used per case 3 can thus be reduced, leading to a reduction in the production costs for systems 1 and facilitating the transport thereof.

The system can be fabricated by assembling two hemispheres, for example, one of them comprising the various solid and/or biological elements intended to be arranged inside the case 3, such as predator mites 2, prey mites 5, substrate 6 and the food source for the prey mites. The inventors have discovered that the growth of predator mites 2 and the capacity of system 1 to spread predator mites 2 are enhanced when the ratio between the number of predator mites 2 and the number of prey mites 5 in the case 3 is comprised between 10⁻⁴ and 1, and preferentially comprised between 4·10⁻³ and 0.5. In particular, this ratio can be controlled at the time of manufacture of the case 3 so as to control the growth kinetics of predator mites 2. Indeed, this ratio allows the quantity of predator mites 2 introduced into each case 3 to be limited.

The inner volume of the case 3 can be partially filled during the manufacture of system 1. Air phase 8 designates the part of the internal volume that is not filled by solid elements. The inventors have also discovered that the growth of predator mites 2 and the capacity of system 1 to spread predator mites 2 are enhanced when the case 3 is not entirely filled by solid elements, i.e., when the volume of air phase 8 is sufficiently high relative to the internal volume of the case 3. In particular, the volume of air phase 8 is greater than 20%, preferentially than 50%, and preferentially greater than 70% of the total internal volume of the case 3.

Different mite species can grow differently in the environment of the case 3.

The case 3 can comprise, for example, the predator mite 2/prey mite 5 pairs described in Table 1.

Preferentially, the predator mites 2 are at least chosen from the phytoseiidae, laelapidae, macrochelidae and cheyletidae families and the prey mites 5 are at least chosen from the carpoglyphidae, pyroglyphidae, glyciphagidae, acaridae, suidasidae and cortoglyphidae families;

Preferentially, the case 3 can comprise the predator mite 2/prey mite 5 pairs described in Table 2.

TABLE 1

| Predator mites 2 | Prey mites 5 |
|---|---|
| | Carpoglyphidae, Carpoglyphus, e.g. Carpoglyphus |
| Hypoaspis, e.g. Hypoaspis angusta, | lactis; |
| Cheyletus, e.g. Cheyletus eruditis, | Pyroglyphidae, Dermatophagoides, e.g. |
| Androlaelaps, e.g. Androlaelaps | Dermatophagoides pteronysinus, |
| casalis, | Dermatophagoides; |
| Laelapidae, e.g. Stratiolaelaps, | Farina, Euroglyphus, e.g. Euroglyphus longior, |
| e.g. Stratiolaelaps scimitus; | Euroglyphus maynei; |
| Gaeolaelaps, e.g. | Pyroglyphus, e.g. Pyroglyphus africanus; |
| Gaeolaelaps aculeifer (Canestrini), | Glycyphagidae, Ctenoglyphinae, e.g. Diamesoglyphus, |
| Androlaelaps, e.g. Androlaelaps | e.g. Diamesoglyphus |
| casalis (Berlese), and/or | Intermedius, Ctenoglyphus, e.g. Ctenoglyphus |
| Macrocheles, e.g. Macrocheles | plumiger, Ctenoglyphus canestrinii, |
| robustulus, | Ctenoglyphus palmifer; Glycyphaginae, e.g. Blomia, |
| Phytoseiides, Amblyseiinae, e.g. | e.g. Blomia freeman, |
| Amblyseius swirskii, Amblyseius | Glycyphagus, e.g. Glycyphagus ornatus, Glycyphagus |
| largoensis, Amblyseius andersoni; | bicaudatus, Glycyphagus privatus, Glycyphagus |
| Neoseiulus, e.g. Neoseiulus | Domesticus; |
| womersleyi, Neoseiulus | Lepidoglyphus, e.g. Lepidoglyphus michaeli, |
| californicus, Neoseiulus cucumeris, | Lepidoglyphus fustifer, Lepidoglyphus destructor, |
| Neoseiulus fallacis, Neoseiulus | Austroglycyphagus, e.g. Austroglycyphagus |
| longispinosus; | geniculatus; Aeroglyphinae, Aeroglyphus, e.g. |
| Iphiseius, e.g. Iphiseius | Aeroglyphus robustus; Labidophorinae, Gohieria, e.g. |
| degenerons; Amblydromalus, e.g. | Gohieria fusca; |
| Amblydromalus lailae, | Nycteriglyphinae, Coproglyphus, e.g. Coproglyphus |
| Amblydromalus limonicus, | stammeri; |
| Amblydromalus manihoti, | Chortoglyphidae, Chortoglyphus e.g. Chortoglyphus |
| Phytoseiulus, e.g. Phytoseiulus | arcuatus, Glycyphaginae, Glycyphagus, Glycyphagus |
| persimilis, Phytoseiulus macropilis | domesticus or Lepidoglyphus destructor; |
| ou Phytoseiulus longipes; | Acaridae, Tyrophagus, e.g. Tyrophagus putrescentiae, |
| Typhlodrominae, Typhlodromips, | Tyrophagus tropicus; |
| e.g. Typhlodromips montdorensis; | Acarus, e.g. Acarus siro, Acarus farris, Acarus gracilis; |
| Euseius, e.g. Euseius ovalis, | Lardoglyphus, e.g. Lardoglyphus konoi, |
| Euseius scutalis, Euseius | Thyreophagus, e.g. Thyreophagus entomophagus; |
| finlandicus, Euseius gallicae, | Aleuroglyphus, e.g. Aleuroglyphus ovatus; |
| Euseius stipulatus, Euseius | Suidasiidae, Suidasia, e.g. Suidasia nesbiti, Suidasia |
| tularensis, Euseius hibisci. | pontifica, Suidasia medanensis, |
| | Tyrolicus casei. |

TABLE 2

| Predator mites 2 | Prey mites 5 |
|---|---|
| Neoseiulus cucumeris | Tyrophagus putrescentiae, |
| | Thyreophagus entomophagus, or |
| | Acarus siro |
| Amblyseius swirskii | Carpoglyphus lactis, |
| | Thyreophagus entomophagus, |
| | Acarus siro, or |
| | Lepidoglyphus destructor |
| Transeius montdorensis | Thyreophagus entomophagus, |
| | Acarus siro, or |
| | Carpoglyphus lactis |
| Amblyseius andersoni | Thyreophagus entomophagus, or |
| | Acarus siro |
| Neoseiulus californicus | Thyreophagus entomophagus, |
| | Acarus siro, or |
| | Lepidoglyphus destructor |

Preferentially, the case 3 comprises Neoseiulus cucumeris predator mites 2 and Tyrophagus putrescentiae prey mites 5.

System 1 can also be used to improve the efficacy of biological control processes in all applications, for example in a field, on a livestock farm or in a greenhouse.

### EXAMPLES

Growth Kinetics for Predator Mites 2

The spread of predator mites 2 is measured for two types of containers: a case 3 in the form of a capsule, conforming to one embodiment of the invention, and a bag known in the prior art. The bags are distinguished from cases 3 of the invention by the porosity to gas and the rigidity of their wall 7 and by the quantity of mites comprised in the container.

To measure the number of predator mites 2 spread by a system, each container (i.e., bag or case 3) is placed under a sheet of transparent plastic in an oasis arena with pollen.

The predator mites 2 are, in this example, Neoseiulus cucumeris and the prey mites 5 are Tyrophagus putrescentiae. The Neoseiulus cucumeris spread are counted every Monday, Wednesday and Friday. The arenas are then washed and put back in place. Four kinetics are measured for each of the experimental conditions. The contents of each arena are washed with 70% ethanol in a 30-mL bottle for liquid counting. Liquid counting consists of pouring each bottle through a 500 μm sieve and a 106 μm sieve, then collecting the components retained by the 106 μm sieve in a beaker in 20 mL or 40 mL of water, depending on the expected density. The beaker is then placed in a magnetic stirrer, and the suspension is mixed. The number of Neoseiulus cucumeris is then counted manually. Six counts are done. The mean of the six counts is multiplied by the dilution factor so as to calculate the number of Neoseiulus cucumeris spread.

The spread of predator mites 2 is measured for two different compositions, each of the compositions being introduced into a case 3 and/or a bag.

The first composition, designated “bag composition”, is a composition typically used in systems of the prior art using a bag as container. The ratio between the number of Neoseiulus cucumeris and the number of Tyrophagus putrescentiae of the composition is equal to 0.04. The composition has a Neoseiulus cucumeris concentration equal to 60,000 L⁻¹ and a Tyrophagus putrescentiae concentration equal to 150,000 L⁻¹. The composition comprises food for prey mites 5 called food type 1A so as to occupy 5% of the total volume. The composition also comprises substrate 6, in particular bran, so as to occupy 85% of the total volume. The ratio between the weight of substrate 6 and the total mite weight is comprised between 5 and 8, or between 500% and 800%.

The second composition is designated by “case composition”. The case composition differs from the bag composition only in that it comprises substrate 6, in particular bran, so as to occupy 10% of the total volume. The ratio between the substrate weight and the total mite weight (i.e., predator mites 2 and prey mites 5) is comprised between 0.06 and 0.08, or between 6% and 8%.

FIG. 2A is a diagram illustrating the kinetics of the number of Neoseiulus cucumeris spread by different spreading systems.

Curve (a) corresponds to the kinetics of the number of Neoseiulus cucumeris spread by a known system of the prior art comprising a bag and a bag composition.

Curve (b) corresponds to the kinetics of the number of Neoseiulus cucumeris spread by a system comprising a bag and a case composition.

Curve (c) corresponds to the kinetics of the number of Neoseiulus cucumeris spread by a system 1 conforming to the invention comprising a case 3 and a case composition.

Curve (d) corresponds to the kinetics of the number of Neoseiulus cucumeris spread by a system comprising a case 3 and a bag composition.

FIG. 2B is a diagram illustrating the kinetics of the ratio between the number of Neoseiulus cucumeris spread by the different spreading systems and the weight of the composition introduced into each of the systems: the number of Neoseiulus cucumeris is thus normalized. This measurement of the number of Neoseiulus cucumeris spread makes it possible to assess efficacy more precisely

Curve (e) corresponds to the kinetics of the ratio between, on the one hand, the number of Neoseiulus cucumeris spread by a system 1 conforming to the invention comprising a case 3 and a case composition, and, on the other hand, the weight of the case composition introduced into system 1

Curve (f) corresponds to the kinetics of the ratio between, on the one hand, the number of Neoseiulus cucumeris spread by a system comprising a case 3 and a bag composition, and, on the other hand, the weight of the bag composition introduced into system.

Curve (g) corresponds to the kinetics of the ratio between, on the one hand, the number of Neoseiulus cucumeris spread by a system known in the prior art comprising a bag and a bag composition, and, on the other hand, the weight of the bag composition introduced into this system.

Curve (h) corresponds to the kinetics of the ratio between, on the one hand, the number of Neoseiulus cucumeris spread by a system comprising a bag and a case composition, and, on the other hand, the weight of the case composition introduced into this system.

The variation between the normalized number of Neoseiulus cucumeris in curve (g) and in curve (f) is less than the variation measured between curve (h) and curve (e): this last variation is caused by a combination of the case packaging and the case composition.

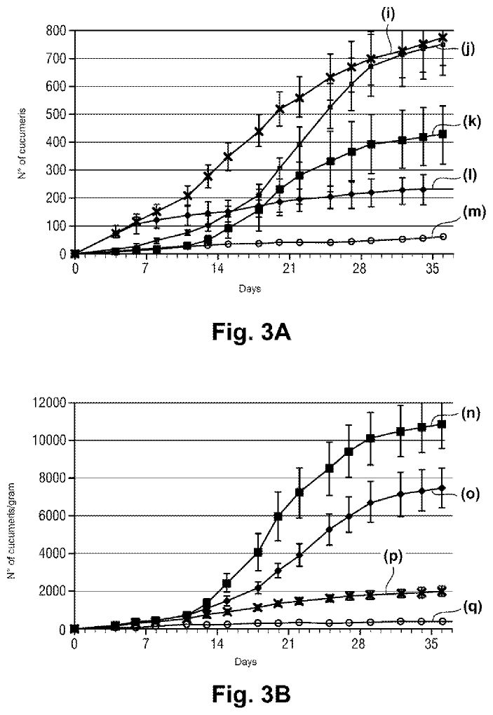

In reference to FIG. 3A and FIG. 3B, a small amount of filling of case 3 leads, surprisingly, to a nonlinear increase in the kinetics of the number of Neoseiulus cucumeris normalized by the weight of the composition introduced into a system 1. In particular, this effect is well known when the volume of air phase 8 of the internal volume of the case 3 is greater than 20% of the internal volume of the case 3, preferentially greater than 50% of the internal volume of the case 3, and preferentially when it is greater than 70% of the internal volume of the case 3. In a complementary manner, this effect is well known for a filling rate of solid phase in the case 3 less than 80%, preferentially less than 50% and preferentially less than 30%.

FIG. 3A is a diagram illustrating the kinetics of Neoseiulus cucumeris spread by a system 1 for different filling rates of a case 3.

Curve (I) corresponds to the kinetics for the number of Neoseiulus cucumeris spread by a system 1, the case 3 having a volume of air phase 8 equal to 50% of the volume of the internal volume of the case 3, i.e., a filling rate of the case 3 equal to 50%.

Curve (j) corresponds to the kinetics for the number of Neoseiulus cucumeris spread by a system 1, the case 3 having a volume of air phase 8 equal to 78% of the volume of the internal volume of the case 3, i.e., a filling rate of the case 3 equal to 12%.

Curve (k) corresponds to the kinetics for the number of Neoseiulus cucumeris spread by a system 1, the case 3 having a volume of air phase 8 equal to 95% of the volume of the internal volume of the case 3, i.e., a filling rate of the case 3 equal to 5%.

Curve (l) corresponds to the kinetics for the number of Neoseiulus cucumeris spread by a system 1, the case 3 having a volume of air phase 8 equal to 15% of the volume of the internal volume of the case 3, i.e., a filling rate of the case 3 equal to 85%.

Curve (m) corresponds to the kinetics for the number of Neoseiulus cucumeris spread by a system 1, the case 3 having a volume of air phase 8 equal to 5% of the volume of the internal volume of the case 3, i.e., a filling rate of the case 3 equal to 95%.

FIG. 3B is a diagram illustrating the kinetics of the ratio between the number of Neoseiulus cucumeris spread by system 1 and the weight of the composition introduced into each system 1, for different filling rates of a case 3.

Curve (p) corresponds to the kinetics for the normalized number of Neoseiulus cucumeris spread by a system 1, the case 3 having a volume of air phase 8 equal to 50% of the volume of the internal volume of the case 3, i.e., a filling rate of the case 3 equal to 50%.

Curve (o) corresponds to the kinetics for the normalized number of Neoseiulus cucumeris spread by a system 1, the case 3 having a volume of air phase 8 equal to 78% of the volume of the internal volume of the case 3, i.e., a filling rate of the case 3 equal to 12%.

Curve (n) corresponds to the kinetics for the normalized number of Neoseiulus cucumeris spread by a system 1, the case 3 having a volume of air phase 8 equal to 95% of the volume of the internal volume of the case 3, i.e., a filling rate of the case 3 equal to 5%.

Curve (q) corresponds to the kinetics for the normalized number of Neoseiulus cucumeris spread by a system 1, the case 3 having a volume of air phase 8 equal to 15% of the volume of the internal volume of the case 3, i.e., a filling rate of the case 3 equal to 85%.

In another example of system 1, the case 3 is a capsule having two orifices 4. A composition comprising 125 Neoseiulus cucumeris, 3000 Tyrophagus entomophagus, 0.240 g of bran (substrate 6) and 0.073 g of food source for prey mites 5 is introduced into the capsule when system 1 is manufactured. The total volume of mites, substrate 6 and the food source introduced into the capsule is equal to 1.483 mL, corresponding to 69% of the total volume of the capsule. The weight of substrate 6 is equal to 1.69 the total weight of predator mites 2 and prey mites 5 in the capsule. The number of Neoseiulus cucumeris per gram of composition is equal to 274 when system 1 is manufactured. The number of Neoseiulus cucumeris per gram of composition spread by the capsule of system 1 after 50 days is equal to 797.

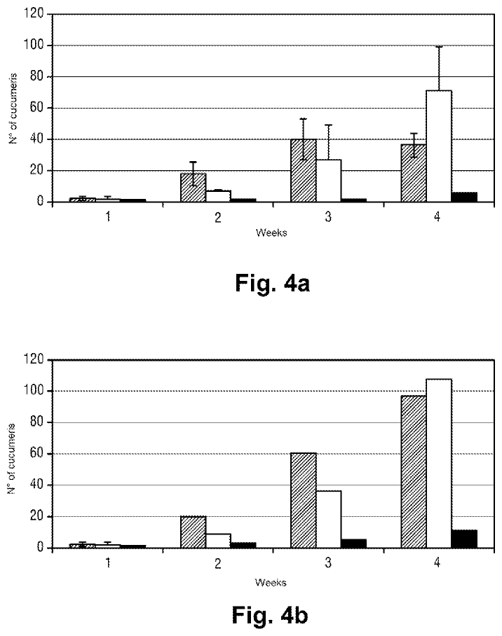

FIG. 4a and FIG. 4b illustrate the spreading kinetics for predator mites by the systems for controlled spread of predator mites conforming to the invention in a greenhouse. The number of Neoseiulus cucumeris is counted, each week, on the on the leaves of cyclamen grown in greenhouses, under three different conditions. The left column for each week on the x-axis is a condition in which a system conforming to the invention is deposited on a cyclamen whose irrigation is provided by watering from the bottom of the pots, in subirrigation. The middle column for each week on the x-axis is a condition in which a system conforming to the invention is deposited on a cyclamen whose irrigation is provided by sprinkler. The right column for each week on the x-axis is a control condition, in which no spreading system is deposited on a cyclamen. FIG. 4a illustrates the measurement of the number of Neoseiulus cucumeris spread on the cyclamen for a given week, i.e., at the time of measuring, while FIG. 4b shows the measurement of a cumulative number of Neoseiulus cucumeris spread on the cyclamen since the deposit of the system conforming to the invention on each cyclamen.

1. System for controlled spread of predator mites,
the system comprising at least one rigid case,
the case having at least one orifice suitable for a passage of predator mites from an inside of the case to an outside of the case,
the system comprising in the case:
predator mites,
prey mites for the predator mites, and
a substrate for the prey mites and the predator mites,
wherein a weight of the substrate is less than twice a total weight of the predator mites and the prey mites in the case.

2. System according to claim 1, wherein a ratio between a number of the predator mites and the prey mites in the case is comprised between 10⁻⁴ and 1.

3. System according to claim 1, wherein the case is partially filled so as to form an air phase, a volume of the air phase being greater than 20% of a total volume of the case.

4. System according to claim 1, wherein the case comprises at least two orifices.

5. System according to claim 1, wherein the case is formed by a wall, a material of the wall being chosen from:
a material comprising fibers, the fibers comprising cellulose, and
polymer material.

6. System according to claim 1, wherein the case has an internal volume comprised between 0.1 mL and 30 mL.

7. System according to claim 1, wherein the orifice has a diameter l comprised between 0.5 mm and 5 mm.

8. System according to claim 1, wherein the case is a capsule.

9. System according to claim 1, wherein the predator mites are at least chosen from the Phytoseiidae, laelapidae, macrochelidae and cheyletidae families and the prey mites are at least chosen from the carpoglyphidae, pyroglyphidae, glyciphagidae, acaridae, suidasidae and cortoglyphidae families.

10. System according to claim 1, wherein the predator mites are at least chosen from Neoseiulus cucumeris, Amblyseius swirskii, Transeius montdorensis, Amblyseius andersoni and Neoseiulus californicus, and wherein the prey mites are at least chosen from Tyrolicus casei, Tyrophagus putrescentiae, Thyreophagus entomophagus, Acarus siro, Carpoglyphus lactis and Lepidoglyphus destructor.

11. System according to claim 1, wherein the predator mites are Neoseiulus cucumeris and wherein the prey mites are Tyrophagus putrescentiae.

12. Manufacturing method for a system according to claim 1,
the method comprising a step of introducing a substrate, predator mites and prey mites into a case,
a weight of the substrate being less than twice a total weight of the predator mites and the prey mites.

13. Manufacturing method according to claim 12, wherein a ratio between a number of the predator mites and the prey mites introduced into the case is comprised between 10⁻⁴ and 1.

14. Use of a system according to claim 1, for biological control. case.

15. Biological control method, comprising a step of depositing into a plant crop at least one system according to claim 1.

16. System according to claim 1, wherein the system also comprises in the case a food source for the prey mites.

17. Manufacturing method according to claim 16, wherein a ratio between a number of the predator mites and the prey mites introduced into the case is comprised between 4×10⁻³ and 0.5.

18. System according to claim 3, wherein a volume of the air phase is greater than 50% of the total volume of the case.

19. System according to claim 3, wherein a volume of the air phase is greater than 70% of the total volume of the case.

20. System according to claim 5, wherein the polymer material is a biopolymer.

21. System according to claim 6, wherein the case has an internal volume comprised between 0.1 mL and 10 mL.

22. System according to claim 6, wherein the case has an internal volume comprised between 0.5 mL and 2 mL.

23. System according to claim 7, wherein the orifice has a diameter/comprised between 1 mm and 2 mm.

24. Manufacturing method according to claim 12, wherein a ratio between a number of the predator mites and the prey mites introduced into the case is comprised between 4×10⁻³ and 0.5.
